(19) United States
(12) Patent Application Publication
(10) Pub. No.: US 2021/0283356 A1
(43) Pub. Date: Sep. 16, 2021

(54) APPLICATION OF UV/FIR TO TREAT INFECTIONS IN THE RESPIRATORY TRACT

(71) Applicants: Ghassan S Kassab, La Jolla, CA (US); Ali Dabiri, San Diego, CA (US)

(72) Inventors: Ghassan S Kassab, La Jolla, CA (US); Ali Dabiri, San Diego, CA (US)

(21) Appl. No.: 17/202,433

(22) Filed: Mar. 16, 2021

Related U.S. Application Data
(60) Provisional application No. 63/008,521, filed on Apr. 10, 2020.
(60) Provisional application No. 62/990,427, filed on Mar. 16, 2020.

(51) Int. Cl. A61M 16/04

(52) U.S. Cl. CPC A61M 16/0402; A61M 2202/206; A61M 2205/053

(57) ABSTRACT

A method of using ultraviolet (UV) radiation to treat an infection in the respiratory system and far infrared radiation (FIR) to treat inflammation. The method uses a light probe inserted into the airway of a patient to apply UV radiation and FIR to a target site in the upper respiratory tract. The UV radiation applied may be broad-spectrum UV radiation or UVC radiation in the 222 nm range. FIR applied may be in the 3-10 μm range. The UV radiation kills any organisms, including COVID-19 and FIR reduces inflammation resulting from infection and subsequent UV treatment. The treatment may be applied to intubated or spontaneously breathing patients and via the oral or nasal passages.

### PRIORITY

The present patent application is related to, and claims the priority benefit of, U.S Provisional Patent Application Ser. No. 62/990,427 filed on Mar. 16, 2020, and is also related to and also claims the priority benefit of, U.S Provisional Patent Application Ser. No. 63/008,521 filed on Apr. 10, 2020, the contents of which are hereby incorporated by reference in their entirety into this disclosure.

### BACKGROUND

COVID-19 belongs to a family of viruses known as coronaviruses. Named for the crown-like spikes on their surfaces, they infect mostly bats, pigs, and small mammals. This family of viruses mutates easily and can transmit from animal to human and from human to human. In recent years, coronaviruses have become a growing player in infectious-disease outbreaks worldwide. Seven strains are known to infect humans, including this new virus (COVID-19) which causes illness in the respiratory tract. Some cause common colds while others, by contrast, rank among the deadliest of human infections: Severe acute respiratory syndrome (SARS), and Middle East respiratory syndrome (MERS).

The COVID-19 virus infects the upper and lower respiratory tract and damages the cells that line the respiratory tract at the alveoli level where the exchange of oxygen and carbon dioxide occurs during respiration. As the virus enters the lung cells, it starts to replicate, destroying the cells. Our body senses all viruses as foreign invaders, which triggers the immune system to contain and control the virus and stop it from replicating itself. The immune system response to the COVID-19 can cause inflammation and destroy lung tissue.

Patients initially develop a fever, cough, and aches, and can progress to suffering from shortness of breath and complications from pneumonia. Other reported symptoms include fatigue, sore throat, headache, and nausea, with vomiting and diarrhea. The end result may be pneumonia which means the air sacs or alveoli in the lungs become inflamed and filled with fluid, making it harder to breathe. These symptoms can also make it harder for the lungs to get oxygen to blood, potentially triggering a cascade of respiratory/cardiac complications. The lack of oxygen leads to more inflammation, which causes more problems in the body resulting in the death of liver and kidney cells and eventually the patient dies.

People of all ages have been infected, but the risk of severe disease and death is highest for older people and those with other health conditions such as heart disease, chronic lung disease, cancer, and diabetes. In certain patients with comorbidities, this condition requires urgent medical attention including the use of a ventilator to stabilize the condition of the patient.

Clinically, patients must be placed on ventilators for weeks as they recover from the viral infection. Recent grim data shows that the majority (over 80%) of patients that are placed on ventilators succumb to the disease and die. It is projected the number of patients that will require respirators dwarfs the number of respirators present available in hospitals and ICUs. Hence, there is a substantial need to reduce the duration of use of respirators by speeding up recovery from the infection. To attain this goal, it is critical to treat high risk patients earlier in the disease stage (in the large airways) to prevent progression of the disease to the smaller airways and alveoli.

There is currently no FDA approved treatment for the COVID-19. Drugs approved for malaria such as chloroquine and antibiotics (e.g., Zithromax Z-PAK, azithromycin) are currently in clinical trial for COVID-19. Even if effective, chloroquine is not without the significant side effects that have been seen when the medication is used for malaria which includes blurred vision, nausea, vomiting, abdominal cramps, headache, diarrhea, bleaching of hair and hair loss. Hence, it is important to devise additional therapies that are local and do not have systemic side effects.

Thus, there is a need for effective treatment of COVID-19 that can be applied both before the patient needs to be intubated and during intubation and additionally does not have systemic side effects.

### BRIEF SUMMARY

This disclosure includes details in connection with the development of a safe and effective treatment to destroy the virus and combat inflammation in the respiratory tract.

The methods and devices noted herein provide therapy in patients to speed up recovery for patients who require or may require ventilators because of an infection in the respiratory system, such as resulting from COVID-19. Clearly, ventilators are in short supply and any reduction in the time needed for ventilators can dramatically impact the healthcare system in this time of crisis and help return people to normal life. The impact of this disclosure to combat this pandemic condition that is paralyzing our country by speeding up recovery of patients affected and reducing the burden on ventilators that are in short demand and the healthcare system cannot be overstated.

An objective of the studies referenced herein is to utilize and optimize an ultraviolet (UV) and/or far infrared radiation (FIR) probe for treatment of COVID-19 in the respiratory tract prior to disease progression to the smaller airways in high risk patients with co-morbidities (e.g., diabetes, hypertension, heart and lung disease, etc.). In another embodiment a UV probe can be used by itself.

As such, in one embodiment, the present disclosure includes disclosure of devices, namely a UV probe, configured for integration with a tracheal tube or to traverse an airway independently, that allows transmission of UV radiation such as UVC or UVA and UVB in the airways that can reach the alveolar sacs where COVID-19 resides. Systems of the present disclosure would therefore include one or more UV probes and one or more other devices or items, such as a tracheal tube, a power source operably coupled to the UV probe to provide power to said UV probe, and the like.

In another embodiment, treatment of the infected patient semi-invasively in a relatively short period can be accomplished by the application of UV light to destroy the virus in the respiratory tract and FIR to treat the inflammation directly in the airways. The UV/FIR fiber optics probe(s) will be introduced through the nasal or oral cavity to reach the respiratory tract. The UV radiation and FIR can be emitted from a single probe or from two probes.

The disclosed treatment is novel in the following ways: 1) Safe and effective in the UV range of operation for short period of time to reduce viral load, 2) Reduce inflammation in the infrared red light range, 3) No drugs with potential systematic harmful side effects into the body, and 4) Potentially lower treatment cost than drugs. The UV/FIR light treatment should not last more than several hours and can also be applied while the patient breathes spontaneously. The impact of this disclosure is to help combat this pandemic condition that is paralyzing our country. The effect of speeding up treatment to reduce the burden on ventilators and the overall effect on the healthcare system which is substantially burdened cannot be overstated. Additionally, the potential for leading to improved outcomes for the many patients who are currently suffering from COVID-19 is great.

The present disclosure includes disclosure of a UV probe, as described herein.

The present disclosure includes disclosure of a system, comprising a UV probe and another device or item, such as a power source, a tracheal tube, and the like.

The present disclosure includes disclosure of methods of treating a viral infection of the lung using a UV probe to emit UV light at or near the location of the virus causing the viral infection in the lung.

The present disclosure includes disclosure of a method, comprising the steps of positioning a UV probe within a trachea or another part of the respiratory system of a mammalian patient so that the portion of the UV probe configured to emit UV light is at or near the location of the virus causing the viral infection in the lung, and operating the UV probe to emit UV light at or near the location of the virus causing the viral infection in the lung to kill some or all of the virus.

The present disclosure includes disclosure of a method, comprising the steps of inserting a tracheal tube within a trachea or another part of the respiratory system of a mammalian patient, positioning a UV probe within the tracheal tube so that the portion of the UV probe configured to emit UV light is at or near the location of the virus causing the viral infection in the lung, and operating the UV probe to emit UV light at or near the location of the virus causing the viral infection in the lung to kill some or all of the virus.

The present disclosure includes disclosure of a method, wherein the UV light emitted by the UV probe is UVC light.

The present disclosure includes disclosure of methods of treating mammalian patients having COVID-19 using ultraviolet light.

The present disclosure also includes apparatuses and methods for treating a respiratory infection using combinations of broad spectrum UV, UVC and FIR.

An exemplary method of treating a respiratory infection comprises the steps of: introducing at least one probe into an airway of a patient; advancing the at least one probe to a target site; and activating the at least one probe such that the at least one probe emits ultraviolet (UV) radiation.

An exemplary method of treating a respiratory infection comprises the steps of: introducing at least one probe into an airway of a patient; advancing the at least one probe to a target site; activating the at least one probe such that the at least one probe emits ultraviolet (UV) radiation and activating the at least one probe such that the at least one probe emits FIR.

An exemplary method of treating a respiratory infection comprises the steps of: positioning a probe within a trachea or another part of the respiratory system of a mammalian patient so that a portion of the probe configured to emit UV radiation is at or near the location of a virus causing the respiratory infection; and operating the probe to emit UV radiation at or near the location of the virus causing the UV radiation to kill some or all of the virus.

An exemplary method of treating a respiratory infection comprises the steps of: positioning a probe within a trachea or another part of the respiratory system of a mammalian patient so that a portion of the probe configured to emit UV and to emit FIR is at or near the location of a virus causing the respiratory infection; and operating the probe to emit UV radiation and FIR at or near the location of the virus causing the UV radiation to kill some or all of the virus.

In the exemplary methods for treating a respiratory infection, the UV radiation emitted may be broad spectrum radiation or may be UVC radiation. In the exemplary methods for treating a respiratory infection, the UVC radiation emitted is in the range of 207-222 nm. In the exemplary methods for treating a respiratory infection, the UVC radiation emitted is in the 222 nm range.

In the exemplary methods for treating a respiratory infection, the FIR emitted is in the 3-10 μm range.

In the exemplary methods for treating a respiratory infection, the UV radiation and FIR are emitted simultaneously.

In the exemplary methods for treating a respiratory infection, the at least one probe is introduced into an airway of a patient further through a tracheal tube.

In the exemplary methods for treating a respiratory infection, the UV radiation is emitted within the airway for a duration of 1-4 hours. In the exemplary methods for treating a respiratory infection, the UV radiation and the FIR are emitted for 1-4 hours.

In the exemplary methods for treating a respiratory infection, the target site is the trachea and specifically the upper trachea.

In another exemplary method for treating a respiratory infection the portion of the probe configured to emit UV radiation and configured to emit FIR is rotated while the probe is operated to emit UV radiation and FIR.

An exemplary system for treating a respiratory infection comprises: a UV radiation source; and at least one probe operably connected to the UV radiation source and configured to traverse the airway of an infected patient and configured to emit UV radiation generated by the UVC radiation source.

An exemplary system for treating a respiratory infection comprises: a UV radiation source, a FIR source; and at least one probe operably connected to the UV radiation source and the FIR source and configured to traverse the airway of an infected patient and configured to emit UV radiation and FIR generated by the UVC radiation source and the FIR source.

An exemplary system for treating a respiratory infection comprises: a UV radiation source, a FIR source; and a single probe operably connected to the UV radiation source and the FIR source and configured to traverse the airway of an infected patient and configured to emit UV radiation and FIR generated by the UVC radiation source and the FIR source.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows the surviving fraction of virus as a function of the incident 222-nm far-UVC dose of exposed H1N1 aerosolized viruses, as measured by the number of focus forming units in incubated epithelial cells relative to unexposed controls, according to an exemplary embodiment of the present disclosure;

FIG. 2 shows dependence of ozone cross section to radiation wavelength, according to an exemplary embodiment of the present disclosure;

FIG. 3 shows a UV fiber optics probe and FIR probe inserted through the nasal cavity and progressed to the trachea, according to an exemplary embodiment of the present disclosure; and

FIG. 4 shows embodiments of UV, FIR and a combined UV/FIR probe according to exemplary embodiments of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration.

Furthermore, wherever feasible and convenient, like reference numerals are used in the figures and the description to refer to the same or like parts or steps. The figures are in a simplified form and not to precise scale.

### DETAILED DESCRIPTION

The present disclosure describes the use of UV radiation to treat infections such as by killing viruses in the lung. The present disclosure describes a UV probe which is, in one embodiment, integrated within a tracheal tube to reach the lung directly through intubation when the patient requires a ventilator. Tracheal intubation, usually simply referred to as intubation, is the placement of a flexible plastic tube through the mouth or nostril into the trachea (windpipe) to maintain an open airway or to serve as a conduit through which to administer certain drugs, or as disclosed herein, a device consistent with the present disclosure.

In addition, non-intubated patients may also be treated by the subject of the present disclosure. The UV and/or FIR probe(s) described herein may be introduced nasally or orally and advanced to the treatment site, usually the upper or lower trachea.

The methods and apparatuses disclosed within can treat patients, high-risk or otherwise, in a relatively short period directly at the site of infection to avoid systemic effects. This treatment is novel in that it: 1) reduces viral load directly in the upper airways before it progress to the smaller bronchi, 2) reduce inflammation in situ in the trachea, 3) provides safe exposure to UV radiation based on the UV range of operation and short exposure time, and 4) avoids medications/chemicals with harmful systemic side effects. This therapy can be used beyond COVID-19 and applies to other viruses and influenza strains that claim tens of thousands of lives of patients at high risk every year.

Data indicates that peak COVID-19 RNA concentrations of up to 5×10⁸ copies per swab were reached before day 5 for infected subjects, all being young to middle-aged professionals without significant underlying disease with mild symptoms (1). COVID-19 was later found in sputum (regurgitated from the smaller airways through coughing) at mean concentrations of 1.2-2.8×10⁶ copies per ml (1); i.e., much higher concentrations than the nasal swabs. Although COVID-19 has striking differences from SARS in that successful live virus isolation from throat swabs is possible, COVID-19 resembles SARS in terms of replication in the lower respiratory tract as a result of progression from the upper respiratory tract. This suggests active virus replication in upper conduction respiratory tract tissues that progress distally to the functional (transport) airways (alveolus). This provides the rationale to focus the radiation intervention in the upper respiratory tract in the earlier stage of the disease to mitigate progression to the smaller airways.

A virus is not a living organism, but consists of RNA covered by a protective layer of lipid (fat), which, when absorbed by the cells of the ocular, nasal or buccal mucosa, changes their genetic code (mutation) and converts them into an aggressor that multiplies. The immune response to the COVID-19 can also destroy respiratory tract tissues and cause inflammation. Since the virus is not a living organism but a RNA strand, it is not killed; it decays on its own. The disintegration time depends on the temperature, humidity and type of material where it lies. The virus is relatively fragile; the only thing that protects it is a thin outer layer of fat and heat melts fat and disintegrates the virus. UV light causes site-specific capsid protein backbone cleavage that inhibits viral genome injection into the host cell.

Ultraviolet Radiation

Scientists have been using ultraviolet (UV) light to kill bacteria, viruses, and other single cell organisms like algae for decades (2). This process is called “ultraviolet germicidal radiation” and works by breaking down the molecular bonds that hold the virus DNA together thereby preventing the virus from multiplying and breaking the life cycle of the virus. Since COVID-19 is a virus, it can be destroyed in the same way (3) and a reduction in viral load along with a reduction in inflammation will lead to speeder recovery.

UV light or UV radiation is classified into three components: Ultraviolet A (UVA), Ultraviolet B (UVB) and Ultraviolet C (UVC). Most of the UV light (98.7%) that reaches us on the earth's surface is of type UVA, in the range of 315-400 nm, and is not affected by the ozone. The remainder (1.3%) is UVB. UVB is mostly absorbed by the ozone layer and it is in the range of 280-315 nm. No UVC rays, in the range of 100-280 nm, reach the earth's surface as UVC is scattered and absorbed by the atmospheric oxygen, nitrogen and ozone. UVC (0%, does not reach the earth's surface) has been found to be safe in a mice study (4).

In test chambers, scientists found 222 nm far-UVC radiation was just as effective as broad-spectrum UV radiation at destroying and deactivating the aerosolized influenza virus (5). It has previously shown that far-UVC light (207-222 nm) can efficiently inactivate bacteria without harm to exposed mammalian skin (6). This is because due to its strong absorbance in biological materials, far-UVC light cannot penetrate even the outer layers of human skin or eye. Since bacteria and viruses are of micrometer or smaller dimensions however, far-UVC can penetrate and inactivate them. Researchers have shown that far-UVC efficiently inactivates airborne aerosolized viruses, with a very low dose of 2 mJ/cm² of 222-nm light inactivating >95% of aerosolized H1N1 influenza virus (7).

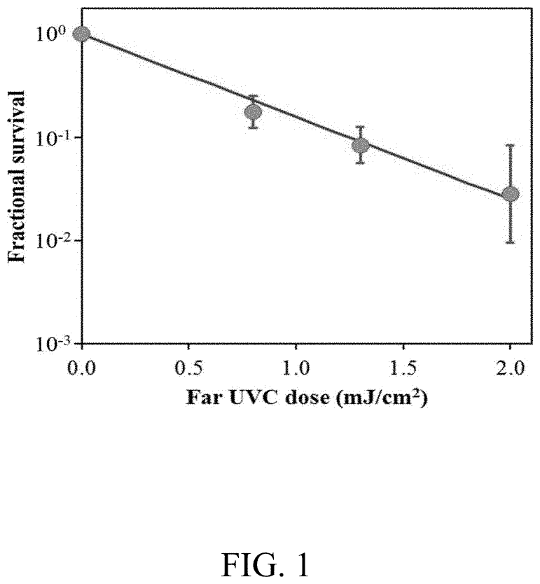

FIG. 1 shows the surviving fraction of virus as a function of the incident 222-nm far-UVC dose, of exposed H1N1 aerosolized viruses, as measured by the number of focus forming units in incubated epithelial cells relative to unexposed controls. Linear regressions showed that the survival results were consistent with a classical exponential UV disinfection model with rate constant k=1.8 cm²/mJ (95% confidence intervals 1.5-2.1 cm²/mJ). The overall model fit was good, with a coefficient of determination, R²=0.95, which suggests that most of the variability in virus survival was explained by the exponential model. The rate constant of 1.8 cm²/mJ corresponds to an inactivation cross-section (dose required to inactivate 95% of the exposed viruses) of D₉₅=1.6 mJ/cm² (95% confidence intervals 1.4-1.9 mJ /cm²).

In one study, the difference in the radiation wavelength in terms of skin injury was investigated (8). The researchers found that UVC radiation is highly germicidal. Exposure to 254 nm-UVC light, however, causes DNA lesions such as cyclobutane pyrimidine dimers (CPD) in human cells, and can induce skin cancer after long-term repeated exposures. It has been reported that short wavelength UVC is absorbed by proteins in the membrane and cytosol, and fails to reach the nucleus of human cells. Hence, irradiation with 222 nm UVC is likely the optimum combination of effective disinfection and biological safety to human cells (8).

In another study, the biological effectiveness of 222 nm UVC was investigated using a mouse model of a skin wound infected with methicillin-resistant Staphylococcus aureus (MRSA) (8). Irradiation with 222 nm UVC significantly reduced bacterial numbers on the skin surface compared with non-irradiated skin. Bacterial counts in wounds evaluated on days 3, 5, 8 and 12 after irradiation demonstrated that the bactericidal effect of 222 nm UVC was equal to or more effective than 254 nm UVC. Histological analysis revealed that migration of keratinocytes which is essential for the wound healing process was impaired in wounds irradiated with 254 nm UVC, but was unaffected in 222 nm UVC irradiated wounds. No CPD-expressing cells were detected in either epidermis or dermis of wounds irradiated with 222 nm UVC, whereas CPD-expres sing cells were found in both epidermis and dermis irradiation with 254 nm UVC. These results suggest that 222 nm UVC light may be a safe and effective way to reduce the rate of surgical site and other wound infections (8) and therefore also safe to use in the elimination of foreign infections.

In terms of safety, UV (A&B) is the most important modifiable risk factor for skin cancer and many other environmentally influenced skin disorders (6). UV also benefits human health, however, by mediating natural synthesis of vitamin D and endorphins in the skin. Therefore, UV has complex and mixed effects on human health. Nonetheless, excessive exposure to UVA&B carries health risks, including atrophy, pigmentary changes, wrinkling and malignancy. In our application of UV, the exposure time is so short that the radiation should not produce any lasting change in the histology of the exposed tissue. It is crucial to design a safe UV system for the patient therapy under short exposure time which is the major goal of this disclosure.

One of the important design requirements in this disclosure is to select a UV radiation bandwidth that is safe for short exposure time (on the scale of hours). UVB and UVC both generate ozone in some region of the wavelength. The same chemical properties that allow high concentrations of ozone to react with organic material outside the body give it the ability to react with similar organic material that makes up the body and potentially cause harmful health consequences. When inhaled, ozone can damage the respiratory tract. Relatively low amounts can cause chest pain, coughing, shortness of breath, and throat irritation. Ozone may also worsen chronic respiratory diseases such as asthma and compromise the ability of the body to fight respiratory infections. People vary widely in their susceptibility to ozone. Healthy people, as well as those with respiratory difficulty, can experience breathing problems when exposed to ozone. Recovery from the harmful effects can occur following short-term exposure to low levels of ozone, but health effects may become more damaging and recovery less certain at higher levels or from longer exposures (9). The band width is selected to minimize the effect of the ozone harmful effects.

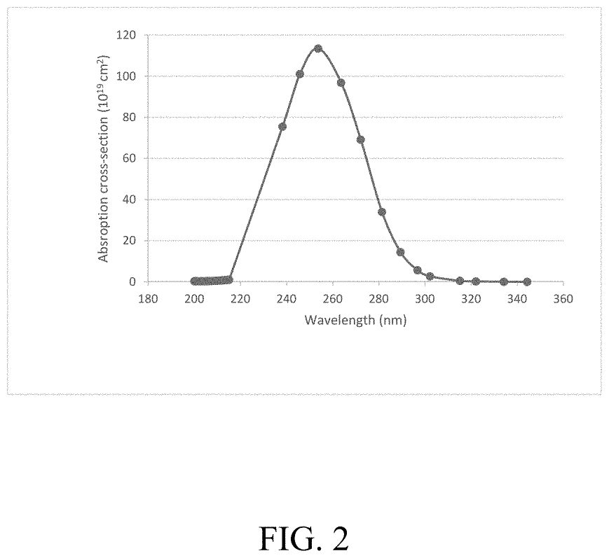

The absorption cross section of ozone changes drastically as function of the UV wavelength (10). For example, in the UVB range, the cross section reduces from around 34×10⁻¹⁹ cm² at 280 nm to less than 1×10⁻¹⁹ cm² at 315 nm at room temperature (14) range. In the UVC range, cross section peaks at 245 nm at 115×10⁻¹⁹ cm² at room temperature. It then drops to 0.3×10⁻¹⁹ cm² at about 200 nm at room temperature. This dependence is shown in FIG. 2. We therefore need to have an optimum design in terms of the wavelength of the UV, exposure time, and the amount of ozone generation. At 315 nm, the cross section of ozone is 3% as much as 280 nm.

Far Infrared Radiation

An additional potential concern may be if more respiratory tract damage is induced by killing the virus in the respiratory tract as an immunopathologic inflammatory reaction with infiltration of proinflammatory cells and mediators. If so, a combination of UV/FIR and some anti-inflammatory drugs may be warranted. Furthermore, it may be argued that reduction of viral load on the airways and respiratory tract may not be as important in the later stages of the disease when the patient is on respirator; i.e., the over-immune response and severe inflammation are the major issues. Regardless, the inflammation is in response to the virus and any reduction in viral load is likely to be important.

Infrared therapy is an effective and safe remedy for pain and inflammation (11). Since infrared therapy enhances and improves circulation in the skin and other parts of the body, it can bring oxygen and nutrients to injured tissues, promoting healing. Other studies indicate that FIR therapy is effective in relieving pain in patients with chronic pain, chronic fatigue syndrome, and fibromyalgia (12). For these reasons, UV radiation in conjunction with FIR is referenced herein for this concept.

As noted herein, we disclose a novel approach to treat the infected high risk patients with co-morbidities (e.g., diabetes, hypertension, heart and lung disease, etc.) earlier in the disease process by the application of UV radiation directly and for a short duration to destroy the virus in the upper respiratory tract and FIR light to reduce the inflammation. The light probe can comprise a single probe configured to emit UV light and FIR or two probes, one configured for FIR and the other for UV light. The light probe(s) is introduced through the nasal cavity or orally to reach the respiratory tract.

The disclosed method inserts the UV/FIR fiber optics probes into the airways earlier in the infection process to reduce the viral load and combat inflammation to mitigate the progression of the etiology of the infection to the lower respiratory tract that impacts gas exchange. UV light having wavelengths in the 280-400 nm range and a short exposure time is used to kill the virus and FIR in the wavelength range of 3-10 μm is used to reduce inflammation. The exposure time can be modified depending on the radiation intensity of the radiation source.

Finally, although two different probes (UV and FIR) can be used, the two can be combined into a single probe once we have validated the current studies. The refinement of the probes is a technological development that can be made easier and more seamless for use in patients.

UV and FIR Probes for Treatment

Exemplary systems for treating infectious diseases in the respiratory system comprise one or more radiation sources capable of generating UV radiation or both UV radiation and FIR emission and are operably connected to at least one probe 10, 12, 14 configured to navigate the airway 22 of the patient and transmit the generated UV radiation and FIR to a target treatment site.

The probes of the current disclosure can comprise any probe sufficiently sized and flexible enough to navigate the airways to reach the treatment target site can be used. Thus the diameter of the probes should be smaller than the airways it will travel through, such as the nasal or oral cavity and the trachea. Where an intubated patient is being treated, the selected probes should fit within the intubation tube. The probes should also be of a sufficient length such that their distal end 16 can reach the target site.

The probes are configured to emit UV radiation and FIR from their distal end 16. The emitted UV radiation can comprise UVA, UVB, UVC, or any combination thereof. In one embodiment, the emitted UV radiation comprises UVA and UVB radiation. In one embodiment, the UV probe 10 is configured to emit radiation in the 280-400 nm range. In another embodiment, the UV radiation comprises UVC radiation, and the UVC radiation is preferably emitted at 222 nm. In another embodiment, the UV radiation comprises far-UVC light (207-222 nm). In another embodiment, the UV radiation comprises UVC radiation at 254 nm.

The FIR probe 12 preferably emits in the 3-10 μm range.

In an exemplary embodiment, the SUPERSPOT MAX 100 Watt UV system manufactured by American Ultraviolet Co (13) including the fiber optics probe is used. The fiber optics outside diameter is about 5 mm and it is 100 cm long where the diameter is small enough to have room for a ventilator. The beam diameter is about 1.8 mm. The energy level can be adjusted as desired. The range of operation is 280-400 nm which is a good compromise for the following reasons: 1) The ozone production is very low (see FIG. 1), and 2) It can heat up the virus locally to disintegrate its membrane, which should destroy the virus. The amount of heat will not damage the tissue since the melting temperature of fat range is 30-40° C. (14). A higher beam diameter (3 mm laser beam) from the same source to access more energy can be used if 4 hours of exposure at lower energy cannot disintegrate the virus. A 3.0 mm fiber optics probe can be used to destroy the virus at certain amounts of exposure time.

Treatment Methods

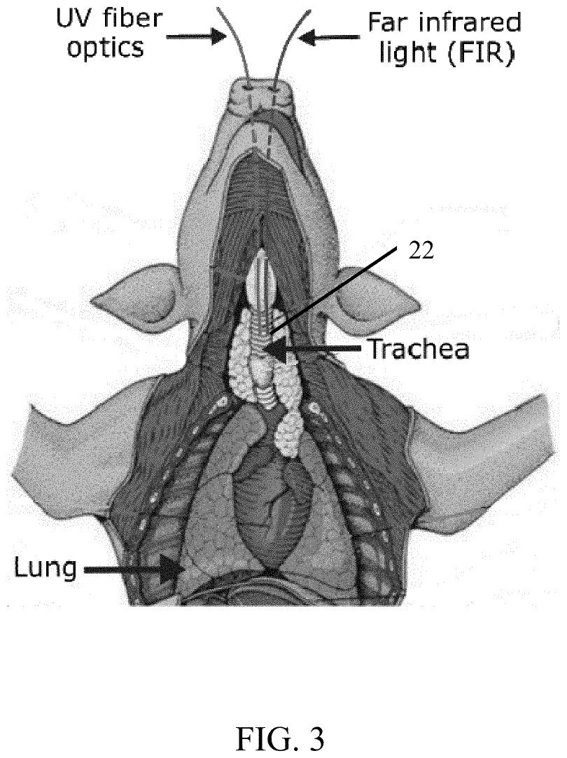

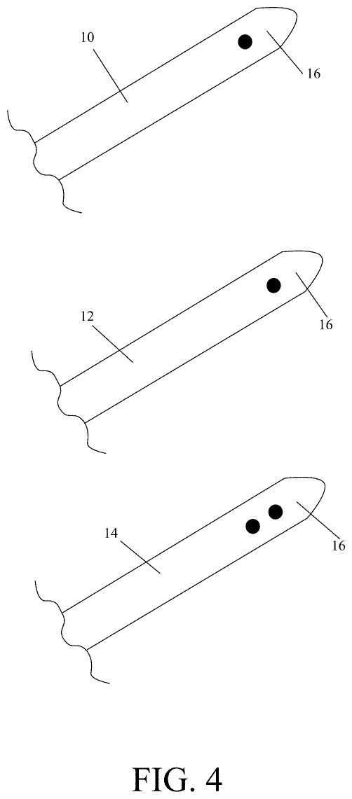

Treatment can be performed utilizing the probes illustrated in FIG. 4. A UV fiber optics probe 10 is inserted through the nasal cavity and progresses to the trachea as depicted in FIG. 3. Similarly, the FIR probe 12 can be inserted either through the adjacent, or same, nostril to also reach the trachea 22. The small diameter probes can be advanced to the region of interest through the use of fluoroscopy. An adult's trachea has an inner diameter of about 1.5 to 2 cm (0.59 to 0.79 in) and a length of about 10 to 11 cm (3.9 to 4.3 in); wider in males than females. It begins at the bottom of the larynx, and ends at the carina, the point where the trachea branches into left and right main bronchi. The inner diameter is smaller for children. The diameter of the fiber optic probes (both UV and FIR) are significantly smaller (3 mm or less) and hence will not interfere with respiration in the relatively larger diameter trachea. In summary, the UV/FIR fiber optics technology has the following advantages. 1) Significantly smaller diameter than trachea, 2) Flexible, and 3) The electrical connections to power the probes are placed outside of the body.

The UV/FIR fiber optics can be inserted through each of the nasal cavities. The UV/FIR probes will be converged on the same segment of the trachea (either upper or lower) to allow transmission of UV/FIR simultaneously. The exposure time of 1 hour is significantly longer than the 30 minutes reported on the bench (12,15) at fiber optics laser power of 10 mw to account for viral disintegration. We also use 4× (four hours) the duration to ensure safety margin and maintained efficacy.

Three exemplary exposure times can include 1, 2 and 4 hours but others may be chosen as desired to kill more viruses or may be chosen dependent on radiation intensity. At each exposure time the radiation intensity may start from 50% of the maximum intensity and then 100%.

A user of the device can rotate the distal end of the unit that emits the UV light to make sure to cover all the exposed surfaces, if needs arise.

The rise in temperature does not significantly affect trachea cells. The viral load is disintegrated at lower values of the exposure time, i.e., reduce viral load. The optimum energy intensity and exposure time can be determined to effectively disintegrate the viral load.

In another embodiment, the patient for treatment has been intubated. In this instance, a dual function probe 14 capable of emitting FIR and UV radiation or two probes, the first emitting UV radiation 10 and the second emitting FIR 12, are introduced into the patient through the intubation tube, which itself may be placed orally or nasally. The probe(s) is advanced to the treatment site and activated to emit UV radiation. If a single probe 14 is used, the probe can be activated to simultaneously emit FIR and UV radiation. If two probes are used, the two probes can be activated such that the patient is exposed to UV radiation and FIR simultaneously. After treatment for a short duration (1-4 hours), the probes are removed

In embodiments utilizing only a UV treatment, only a single probe capable of emitting UV radiation is required. FIR is not administered, but the rest of the procedure is similar to a procedure performed with both UV and FIR.

During treatment, FIR in the in the 3-10 μm range is emitted from the probes. Preferably, UVC radiation is emitted and preferably in the 222 nm range. In another embodiment, far UVC light is emitted in the range of 207-222 nm. Alternatively, broad spectrum UV may be emitted during treatment.

After treatment is complete, the probes may be removed.

While various embodiments of devices, systems, and methods to kill coronavirus in the lung using ultraviolet light and far infrared ray have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

For example, although the specification refers to treating COVID-19, it is within the scope of the disclosure that other types of coronavirus or, in general, other viruses and diseases susceptible to UV destruction could be treated by the methods and apparatus disclosed. It is also envisioned that other locations in the body could be treated. For instance, the trachea is specifically referred to, but other portions of the lungs or other luminal organs could be the target for treatment.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and remain within the scope of the present disclosure.

### REFERENCES

1. R. Wölfel, et al.,Virological assessment of hospitalized patients with COVID-2019, Nature, Apr. 1, 2020. Nature https://doi.org/10.1038/s41586-020-2196, 2020.

2. Ultraviolet germicidal irradiation—Wikipedia, accessed March 2020.

3. https://findme10.com/best-coronavirus-uv-light/.

4. S. Saadati, study of ultraviolet C light penetration and damage in skin, Sahlgrenska Academy Department of Radiation Physics, Columbia University Medical Center, University of Gothenburg, 2016.

5. https://www.upi.com/Science News/2018/02/09/UV-light-can-kill-airborne-flu-virus-study-finds/3081518201355/.

6. J. D'Orazio et al., UV Radiation and the Skin, Int J Mol Sci., 14(6): 12222-12248, 2013.

7. D. Welch et al., Far-UVC light: A new tool to control the spread of airborne-mediated microbial diseases, Scientific Reports, 8, Article number: 2752, 2018.

8. K. Narita, Disinfection and healing effects of 222-nm UVC light on methicillin-resistant Staphylococcus aureus infection in mouse wounds, J. of Photochemistry and Photobiology B: Biology, 178: 10-18, 2018.

9. https://www.ozonesolutions.com/knowledge-center/ozone-health-effects.html

10. J. MALICET et al., Ozone UV Spectroscopy. II. Absorption Cross-Sections and Temperature Dependence, Journal of Atmospheric Chemistry 21: 263-273, 1995.

11. Shanshan Shui, Xia Wang, John Y Chiang, and Lei Zheng, Far-infrared therapy for cardiovascular, autoimmune, and other chronic health problems: A systematic review, Exp Biol Med (Maywood), 240(10): 1257-1265, 2015.

12. Everett M. Lautin, and Suzanne M. Levine, Use of Near-Infrared Light Source to Treat Pain, Practical Pain Management, 2012.

13. www.auvco.com.

14. KNUT SCHMIDT-NIELSEN, Melting Points of Human Fats as Related to their Location in the Body, First published: June 1946, https://doi.org/10.1111/j.1748-1716.1946.tb00372.

15. Jui-Chih Chang et al., Far-infrared radiation protects viability in a cell model of Spinocerebellar Ataxia by preventing polyQ protein accumulation and improving mitochondrial function, Scientific Reports, 6:30436, 2016.

1. A method of treating a respiratory infection comprising the steps of:
introducing at least one probe into an airway of a patient;
advancing the at least one probe to a target site; and
activating the at least one probe such that the at least one probe emits ultraviolet (UV) radiation.

2. A method of treating a respiratory infection as in claim 1, further comprising the steps of activating the at least one probe such that the at least one probe emits far infrared (FIR) radiation.

3. A method of treating a respiratory infection as in claim 1, wherein the UV radiation emitted is broad spectrum radiation.

4. A method of treating a respiratory infection as in claim 1, wherein the UV radiation emitted is UVC radiation.

5. A method of treating a respiratory infection as in claim 4, wherein the UVC radiation emitted has a wavelength of 222 nm.

6. A method of treating a respiratory infection as in claim 2, wherein the FIR emitted is in the 3-10 μm range

7. A method of treating a respiratory infection as in claim 1, wherein the step of introducing at least one probe into a airway of a patient further comprises the step of introducing at least one probe through a tracheal tube.

8. A method of treating a respiratory infection as in claim 1, further comprising the step of emitting UV radiation within the airway for a duration of 1-4 hours.

9. A method of treating a respiratory infection as in claim 1, wherein the target site is the upper trachea.

10. A method of treating a respiratory infection, comprising the steps of:
positioning a probe within a trachea or another part of the respiratory system of a mammalian patient so that a portion of the probe configured to emit UV radiation is at or near the location of a virus causing the respiratory infection;
operating the probe to emit UV radiation at or near the location of the virus causing the UV radiation to kill some or all of the virus.

11. A method of treating a respiratory infection as in claim 10, wherein the portion of the probe configured to emit UV radiation is also configured to emit FIR and the method further comprises the step of operating the probe to emit FIR.

12. A method of treating a respiratory infection as in claim 11, wherein the UV radiation emitted is UVC radiation.

13. A method of treating a respiratory infection as in claim 12, wherein the UV radiation emitted is in the range of 207-222 nm.

14. A method of treating a respiratory infection as in claim 13, wherein the UV radiation emitted has a wavelength of 222 nm.

15. A method of treating a respiratory infection as in claim 11, further comprising the step of emitting UV radiation and FIR simultaneously.

16. A method of treating a respiratory infection as in claim 11, wherein the UV radiation and the FIR are emitted for 1-4 hours.

17. A method of treating a respiratory infection as in claim 11, further comprising the step of rotating the portion of the probe configured to emit UV radiation and configured to emit FIR while the probe is operated to emit UV radiation and FIR.

18. A system for treating a respiratory infection comprising:
a UV radiation source; and
at least one probe operably connected to the UV radiation source and configured to traverse the airway of a infected patient and configured to emit UV radiation generated by the UVC radiation source.

19. A system for treating a respiratory infection as in claim 18, further comprising a FIR source and wherein the at least one probe is operably connected to the FIR source and configured to emit FIR generated by the FIR source.

20. A system for treating a respiratory infection as in claim 18, wherein the at least one probe comprises a single probe configured to emit both FIR and UV radiation.
